(19) United States
(12) Patent Application Publication
(10) Pub. No.: US 2023/0094850 A1
(43) Pub. Date: Mar. 30, 2023

(54) Contact lens insertion apparatus

(71) Applicants: Sandra Rita Hunter, Cambridge (CA); Terrance Rodney Hunter, Cambridge (CA)

(72) Inventors: Sandra Rita Hunter, Cambridge (CA); Terrance Rodney Hunter, Cambridge (CA)

(21) Appl. No.: 17/300,442

(22) Filed: Jul. 1, 2021

(51) Int. Cl. A61F 9/00

(52) U.S. Cl. CPC A61F 9/0061

(57) ABSTRACT

An apparatus facilitates insertion of contact lenses into a user’s eyes. The apparatus includes a conical bellows-like collapsible stand with different diameter steps having an opening at the top for receipt of a simple lens insertion tube which receives the convex surface of a lens in a cup. Positioned within the open interior of the housing are one or more LEDs, which may be of different colors, which may be controlled by a wireless remote control. Alternatively the stand can have an open bottom which can be placed over a light source of a cellphone.

### BACKGROUND AND SUMMARY OF THE INVENTION

Contact lenses are commonly used by millions of people to facilitate proper sight without the need for glasses. Typically, a user installs or removes a contact lens in/from an eye using one or more fingers. Usually, the contact lens is balanced on one finger and one or both lids of the eye are pulled back from the eye. A few drops of saline or other artificial eye fluid are dropped onto the concave surface of the lens. The lens is then gently pushed against the eye, where forces of cohesion and adhesion will cause the lens to attach to the eye and float on the thin layer of fluid on the surface of the eyeball. This procedure can be difficult and stressful for some people since it requires manual dexterity and hand and eye stability to ensure that the contact lens is brought into sufficient proximity to allow attachment to the eye surface without causing accidental or painful contact with the cornea or sclera. Likewise, removal requires a steady hand and the ability to pinch or squeeze the contact to dislodge it from the surface of the eye.

Because of potential difficulties in insertion and removal of contacts many devices have been proposed, or marketed, designed to assist with the contact lens insertion and removal processes. Many utilize suction forces to hold the contact while inserting it or to pull the contact from the eye during removal; however, if the contact is not immediately released upon adhering to the eye, the suction force can cause painful contact with the cornea or sclera during installation and may even remove the contact instead of leaving it in place. It is also possible that the contact lens forms a bond with the eye surface. Further, the insertion device may have to be moved toward the eye by hand, and if the user’s hand is unsteady the device may be also as difficult to use as no device at all.

One way to enhance the functionality of a contact lens insertion device is to use a stand on which the lens is placed so that the user can move her/his face toward a steady support structure rather than relying on the steadiness of the user’s hand. Prior art stands include a small simple base (USP 3,791,689), a large rigid structure facilitating use of a squeezable plunger inserter (See-Green® Lens Inserter), a large complex device that uses a rack and pinion to move a cup holding a contact lens up and down (USP 9,788,997), or a mirror and support tube (USP 10,182,938). While all of these devices are functional each has one or more drawbacks associated therewith including storage difficulties, contact lens solution contamination, and less than ideal operative association with a light source since the use of a light source is desirable with all insertion devices. Also, the actual insertion device may itself be complicated and/or expensive to replace.

According to the present invention, a contact lens insertion apparatus is provided that has many advantages compared to prior art devices, and seeks to optimize the utility of the apparatus for the insertion of contact lenses.

The apparatus according to the invention uses a simple inexpensive and readily replaceable insertion tool, and an adjustable collapsible stand that is easy to store and adapt to individual users and directs spilled contact solution away from the inserter and stand. The invention also mounts a unique light source within the stand in a desirable manner, or allows the stand to be used with a portable light source such as a cellphone. In general the apparatus according to the invention facilitates easy and versatile insertion of contact lenses into a user’s eyes.

In one example, the insertion tool is a simple tube of silicone with a cup at the top capable of receiving the convex portion of a contact lens therein. There is no light source in the tool itself, suction capability, or other complicating feature so that the insertion tool per se is inexpensive and easy to replace.

In this example, the basic features of the stand are a collapsible conical housing having an open top for removably receiving the insertion tool, with one or more differently colored light emitting diodes (LEDs) removably positioned within the interior of the stand, preferably covered by a transparent protector. The collapsible conical construction of the stand allows adjustability, a low volume for ease of storage, durability with no realistic chance of breakage, and a shape that directs any spilled contact lens solution away from the housing.

The apparatus also includes a control for activating the LEDs, such as a wireless remote control. The color of LED used is easily selected to fit the user’s particular needs or desire, as is its brightness. Light from one or more of the LEDs shines up through the stand and through the interior of the insertion tool to facilitate placement of a contact lens on the insertion tool into the user’s eye. There is no vacuum source for holding the contact lens on the cup of the insertion tool since a vacuum or suction source may have the disadvantages mentioned above.

More specifically, according to one aspect of the invention there is provided an apparatus facilitating insertion of a contact lens in a user’s eye. The apparatus comprises: A collapsible stand having at least one high volume use configuration and a low volume storage configuration, with an open interior and an opening at a top portion thereof. A tubular insertion tool having a cup at a first end thereof shaped to receive the convex surface of a contact lens therein, and a second end thereof shaped and dimensioned to fit in and be securely held by the opening at a top portion of the stand. At least one light source positioned within the open interior of the stand so that light from the at least one light source passes upwardly through the open interior of the stand, through the opening at a top portion of the stand, and through the tubular insertion tool when received by the stand opening; and a control for controlling operation of the at least one light source.

Preferably the stand has several use locations positioning the top portion opening at adjustable heights. This can be accomplished by constructing the stand so that it has a substantially conical bellows-like construction with different diameter steps, e. g. from a material selected from the group consisting essentially of molded nitrile, neoprene, silicone, and natural rubber, and plastics having properties comparable to rubber.

Preferably the tubular insertion tool consists of a simple rubber or plastic (desirably silicone rubber) tube with an integrally molded cup at the first end thereof.

The at least one light source preferably comprises a plurality of differently colored (e. g. red, green, yellow, white, blue) LEDs mounted in a base connected to the bottom of the stand. Within the base there may be a transparent protective material to preclude the LEDs from being contacted by spilled contact lens solution. The base may include a hollow housing having a support structure therein, the light emitting diodes mounted on a first face of the support structure and a casing for one or more batteries mounted on a second face of the support structure, opposite the first face.

The invention also contemplates the tubular insertion tool mounted in the stand top opening and in combination with a contact lens having the convex surface thereof received by the insertion tool cup (and perhaps contact lens solution received by the concave surface of the contact lens).

According to another aspect of the present invention there is provided an apparatus facilitating insertion of a contact lens in a user’s eye that comprises: A collapsible stand having a substantially conical bellows-like construction of different diameter steps, with an open interior and an opening at a top portion thereof; and a tubular insertion tool having a cup at a first end thereof shaped to receive the convex surface of a contact lens therein, and a second end thereof shaped and dimensioned to fit in and be securely held by the opening at a top portion of the stand. In this aspect of the invention the stand may be open at the bottom thereof so as to be positionable over a portable light source so that light from the portable light source passes through the open interior of the stand, and through the tubular insertion tool to a contact lens supported by the insertion tool cup. The apparatus may be in combination with a cellphone having a light source, the open bottom of the stand positioned over the cellphone light source. Alternatively, the stand may have a bottom connected to a base and the base includes at least one light source and at least one battery. The at least one light source is positioned so that light emanating therefrom passes upwardly through the open interior of the stand, and through the tubular insertion tool to a contact lens supported by the insertion tool cup.

According to a further aspect of the invention there is provided a contact lens tubular insertion tool consisting of a simple silicone rubber tube with an integrally molded cup, shaped at a first end thereof to receive the convex surface of a contact lens therein. The tubular insertion tool may be provided in combination with a contact lens having the convex surface thereof received by the insertion tool cup (and perhaps contact lens solution received by the concave surface of the contact lens).

It is the primary object of the present invention to provide a practical, versatile, apparatus facilitating insertion of a contact lens into a user’s eye. This and other objects of the invention will become clear from a detailed description of the invention, and from the appended claims.

### BRIEF DESCRIPTION OF THE DRAWINGS

FIG. 1 is a top perspective view showing basic components of an exemplary contact lens insertion apparatus according to the invention from the exterior thereof;

FIG. 2 is a disassembled view of two of the major components of the apparatus of FIG. 1 with the stand in a collapsed condition;

FIG. 3 is a side exploded view showing the apparatus of FIGS. 1 & 2 along with a contact lens to be associated therewith;

FIG. 4 is a bottom perspective exploded view of the apparatus of FIGS. 1-3;

FIG. 5 is a top perspective view of the apparatus of FIGS. 1-4 in combination with a wireless remote control;

FIG. 6 is a top perspective exploded view of a second embodiment of an exemplary lens insertion apparatus according to the present invention; and

FIG. 7 is a side cross-sectional view of the apparatus of FIG. 6 in operation.

### DETAILED DESCRIPTION OF THE DRAWINGS

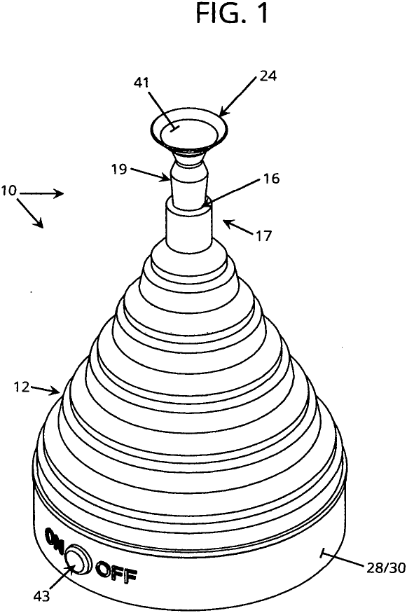

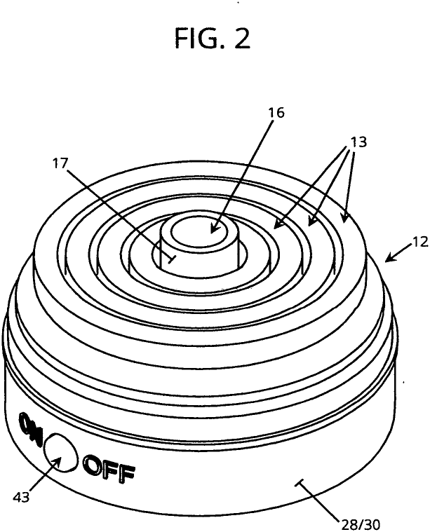

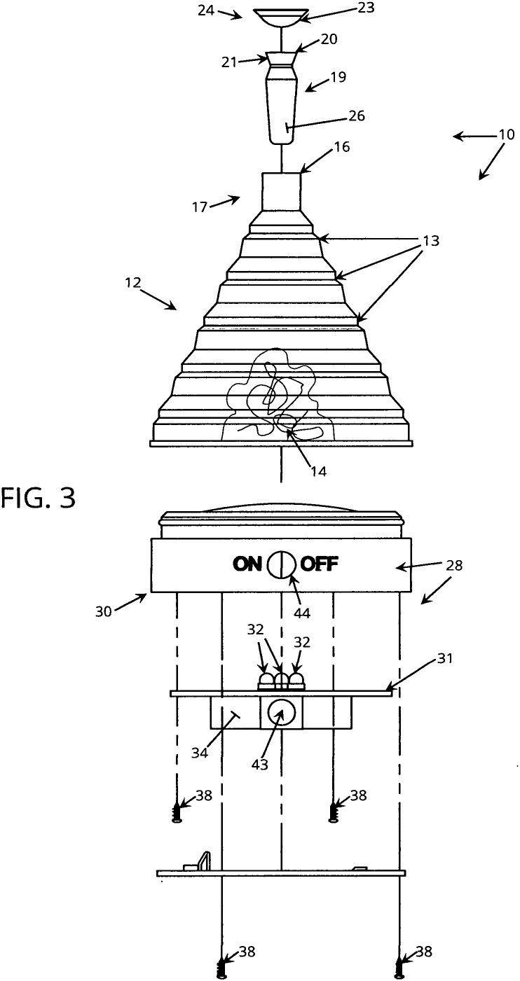

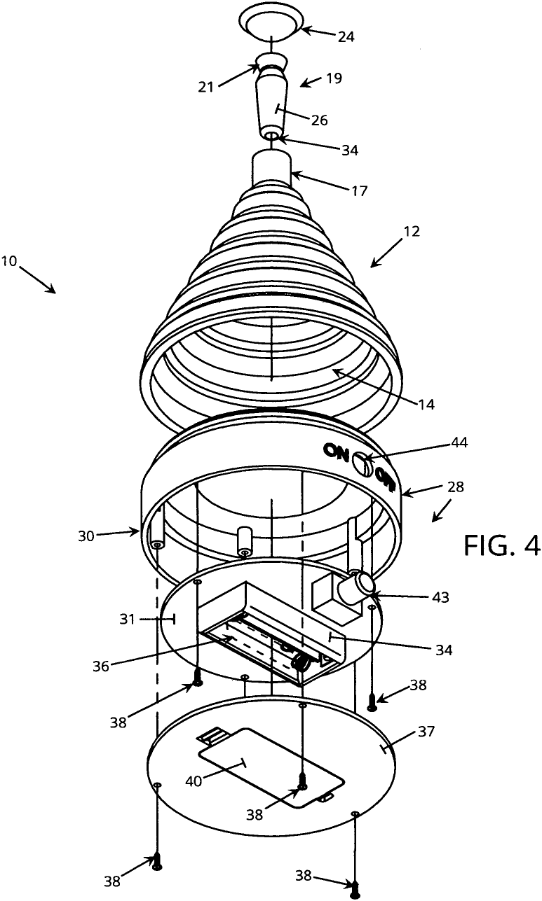

An apparatus according to a first exemplary embodiment of the present invention is shown generally by reference number 10 in FIGS. 1-5. The basic components visible in FIG. 1 are: a collapsible stand 12 having at least one high volume use configuration (as seen in FIG. 1) and a low volume storage configuration (as seen in FIG. 2) preferably with different diameter steps 13, with an open interior 14 (FIG. 4) and an opening 16 (FIGS. 1-3 & 5) at a top portion 17 thereof; a tubular insertion tool 19 (FIGS. 1 and 3-7) having a cup 20 at a first end 21 thereof shaped to receive the convex surface 23 of a conventional contact lens 24 (FIGS. 3 & 4) therein, and a second end 26 (FIGS. 3, 4, 6, & 7) thereof shaped and dimensioned to fit in and be securely but removably held by the opening 16 at the top portion 17 of the stand 12; and a base 28.

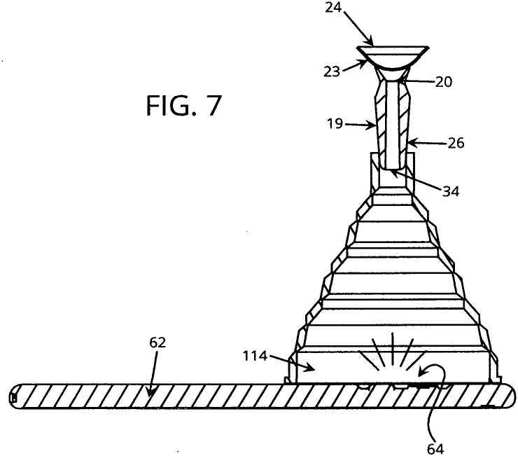

Not seen in FIGS. 1 & 2, but visible in both FIGS. 3 & 4, are the components of the base 28, including a hollow housing 30 containing at least one light source, preferably at least one light emitting diode (see the three LEDs 32 in FIG. 3). When the apparatus 10 is assembled the LEDs 32 are positioned within the open interior 14 of the stand 12 so that light from the one or more LEDs 32 passes upwardly through the open interior 14 of the stand, through the opening 16 at the top portion 17 of the stand 12, and through the open interior (visible at 34 in FIG. 7) of the tubular insertion tool 19 when received by the stand opening 16. The LEDs 32 are preferably mounted on one face of a support 31 (FIGS. 3 & 4). The support 31 on a second, opposite, face also mounts a battery housing 34 (FIG. 4) which mounts one or more conventional batteries (e. g. AA or AAA alkaline batteries), shown in dotted line at 36 in FIG. 4.

A bottom plate 37 (FIG. 4) closes the bottom of the hollow housing 30 while still allowing easy access to the battery(ies) 36 via the readily opened and closed flap 40. Fasteners (e. g. screws) 38 connect the bottom plate to the hollow housing 30, holding the elements 30/31/37 together.

A transparent impermeable plastic film or sheet, shown schematically at 39 in FIGS. 3 & 4, may be provided to cover the LEDs 32 to protect them from spilled or leaking conventional contact lens solution, such as when the solution is received by the concave surface 41 (FIG. 1) of the contact lens 24.

The LEDs 32, battery 36, and any other electrical components, are connected together by conventional circuitry (not shown), and are controlled by a control device, which may have a wide variety of configurations. As one example of the control FIGS. 1-4 illustrate an on/off button 43 which is provided as part of the electrical components mounted by the support 31 to control the energization of one or all of the LEDs 32. The button 43 extends through a hole 44 (FIGS. UES 3 & 4) in housing 30 when the apparatus 10 is assembled (as in FIG. 1).

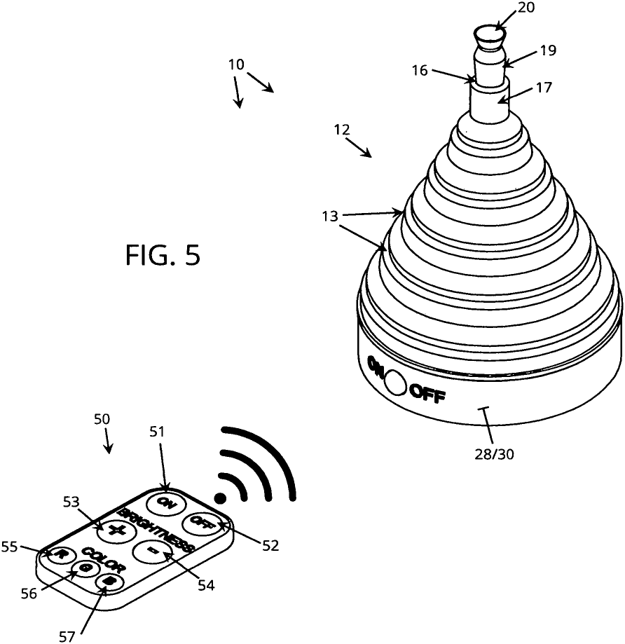

It is desirable that a plurality of LEDs 32 of different color be provided, such as red, green, yellow, white, and/or blue. The different colors are not just for aesthetic purposes since some users find a particular color more suitable for facilitating lens 24 insertion than others. While the desirability of using a light source to help in inserting a lens 24 has been known for decades (e. g. see USPs 3,304,113 and 3,791,689) heretofore no one has recognized that different colored lights may be more helpful to different users. In the exemplary embodiment of FIG. 5 red, green, and blue LEDs 32 may be provided and only one, or only two, or all three, may be operated at the same time. The circuitry associated with the button 43 may provide for one depression to energize the red LED 32, a second depression to energize the green, a third to energize the blue, a fourth to energize all three at once, and a fifth to turn all of the LEDs 32 off.

As seen in FIGS. 1-4, the stand 12 preferably has a construction that allows adjustment of the height of the opening 16, and tool 19 positioned therein, from the surface (e. g. a table top) on which the base 28/30 of the stand 12 sits. As illustrated in the drawings, the stand 12 preferably has the configuration of a substantially conical bellows-like device, having steps 13 of decreasing diameter from the base 28/30 to the top 17 (FIGS. 1-4). The conical nature not only allows for easy collapse to the low volume configuration for stand 12 illustrated in FIG. 2 but helps direct spilled contact lens solution away from the apparatus 10, and makes precise adjustment of the position of opening 16 from the base 28/30 simple. While a wide variety of materials may be used to produce the stand 12, preferably the stand 12 is molded of a material selected from the group consisting essentially of nitrile, neoprene, silicone, and natural rubbers, and plastics having properties comparable to those rubbers.

The insertion tool 19 is desirably uncomplicated; it preferably consists of a simple unitary rubber or plastic (desirably silicone rubber) tube (see FIG. 7) with an integrally molded cup 20 at the first end 21 thereof so that the tool 19 is devoid of a light source, vacuum or suction causing or facilitating element, or any other complicating structure. Thus the tool 19 is straightforward and easy to produce, is relatively inexpensive, and may be readily replaced.

In a typical manner of use of the apparatus 10, the user deploys the conical bellows-like stand 12 from its low volume storage position of FIG. 2 to the use position of FIG. 1, deploying as many steps 13 thereof as necessary to position the opening 16 at the desired distance from the surface on which the base 28 sits. The housing 30 including the LEDs 32 and battery(ies) 36 is positioned in the open interior 14 of the stand 12, held in place by plate 37 and screws 38, and the insertion tool 19 second end 26 is inserted into the opening 16 so that the cup 20 is on top of the tool 19. A conventional contact lens 24 is positioned in the cup 20, with the convex surface 23 of the lens 24 received by the cup 20. Conventional contact solution is dripped or squirted on the concave surface 41 of the lens 24. One or more of the LEDs 32 are then energized using the button 43.

With the contact lens 24 and solution so positioned, the user moves her/his eye toward the lens 24 until the solution and lens 24 come in contact with the user’s eye, thereby inserting the lens 24 into the eye. The user then withdraws her/his head and repeats the process for her/his second eye.

In a more sophisticated and easily useable embodiment, as shown in FIG. 5, the control for the apparatus 10 instead of or in addition to the button 43 comprises a wireless remote control device 50. Device 50 is utilized to control energization of the LEDs 32 by connecting them to, or disconnecting them from, the battery(ies) 36. Mounted by the support 31 are conventional electrical and electronic components that cooperate with the elements 32, 36 to allow control of the operation and brightness of the LEDs 32 by the remote 50.

The remote 50 has on and off buttons, 51, 52, respectively; brightness increasing and decreasing buttons, 53, 54, respectively; and buttons 55, 56, and 57, respectively, for energizing three differently colored LEDs 32 (e. g. red, green, and blue, although any other suitable colors, including white, may be used). One, two, or all three of the buttons 55-57 may be activated at the same time.

The FIG. 5 embodiment, with the remote control 50, allows easy operation of the apparatus 10 by the user, with maximum flexibility and a minimum of difficulty.

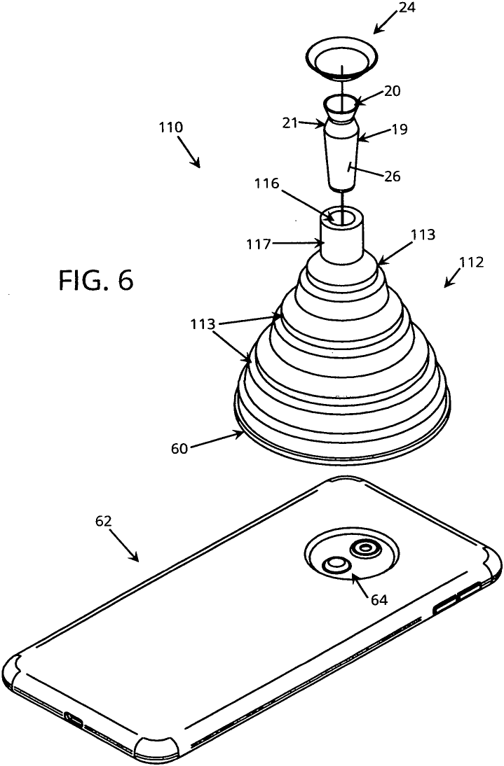

FIGS. 6 & 7 show another exemplary embodiment of an apparatus according to the invention which is more simple than the apparatus 10 and is specifically designed for ease of use during travel. In FIGS. 6 & 7 components comparable to those in the other embodiments are shown by the same reference numeral only preceded by a “1” (e. g. the general apparatus is “110” instead of “10”).

The collapsible stand 112 having steps 113 is typically of a smaller diameter than the stand 12, and perhaps a smaller height (e. g. with less steps 113). The collapsible stand 112 also has an open bottom 60, with no base 28, so that the open interior 114 (FIG. 7) is easily accessible. The insertion tool 19 is (except possibly for size) identical to that in the other embodiments and therefore it, and the various components thereof, are shown by the same reference numerals as in the other drawings.

The light source for the apparatus 110 is a conventional cellphone, shown schematically at 62, in FIGS. 6 & 7. The cellphone 62 – as is conventional in many cellphones – has a white light source (e. g. LED), shown schematically at 64 in the drawings, which is typically configured to be used as a flashlight. The operation of the apparatus 110 is similar to that of the apparatus 10 except that the open bottom 60 of the stand 112 is placed over the portion of the cellphone 62 containing the light source 64 (see FIG. 7) so that the light from source 64 shines upwardly through the open interior 114 of stand 112, the tubular interior 34 of the insertion tool 19, and to the contact lens 24 with solution mounted on the cup 20 of the tool 19.

Thus, it will be seen that the apparatus 110 is very portable (especially when the stand 112 is collapsed like that shown in FIG. 2 for stand 12) and can be used anywhere with the user’s conventional cellphone 62 as the light source 64.

While the invention has been herein shown and described in what is presently conceived to be the most practical and preferred embodiments thereof it is to be understood that the invention is to be interpreted as broadly as the appended claims allow to cover all equivalent structures and apparatus, limited only by the prior art.

What is claimed is:

1. Apparatus facilitating insertion of a contact lens in a user’s eye, comprising:
a collapsible stand having at least one high volume use configuration and a low volume storage configuration, with an open interior and an opening at a top portion thereof;
a tubular insertion tool having a cup at a first end thereof shaped to receive the convex surface of a contact lens therein, and a second end thereof shaped and dimensioned to fit in and be securely yet removably held by said opening at a top portion of said stand;
at least one light source positioned within said open interior of said stand so that light from said at least one light source passes upwardly through said open interior of said stand, through said opening at a top portion of said stand, and through said tubular insertion tool when received by said stand opening; and
a control for controlling operation of said at least one light source.

2. Apparatus as recited in claim 1 wherein said stand has several use positions positioning said top portion opening at adjustable heights.

3. Apparatus as recited in claim 2 wherein said stand has a substantially conical bellows-like construction of different diameter steps.

4. Apparatus as recited in claim 3 wherein said stand is made of a material selected from the group consisting essentially of molded nitrile, neoprene, silicone, and natural rubbers, and plastics having properties comparable to those rubbers.

5. Apparatus as recited in claim 1 wherein said tubular insertion tool consists of a simple rubber or plastic tube with an integrally molded cup at said first end thereof.

6. Apparatus as recited in claim 1 further comprising a base operatively connected to said stand at a bottom portion thereof; and wherein said at least one light source comprises a plurality of differently colored light emitting diodes mounted in said base.

7. Apparatus as recited in claim 6 wherein said light emitting diodes in said base are covered by a transparent protective material to preclude said light emitting diodes from being contacted by leaking or spilled contact lens solution.

8. Apparatus as recited in claim 7 wherein said base includes a hollow housing having a support structure therein, said light emitting diodes mounted on a first face of said support structure and a casing for one or more batteries mounted on a second face of said support structure, opposite said first face.

9. Apparatus as recited in claim 1 wherein said tubular insertion tool is mounted in said stand top opening and in combination with a contact lens having the convex surface thereof received by said insertion tool cup.

10. Apparatus as recited in claim 1 wherein said control comprises a wireless remote control for controlling the operation, including the brightness, of said at least one light source.

11. Apparatus as recited in claim 3 wherein said tubular insertion tool consists of a simple rubber or plastic tube with an integrally molded cup at said first end thereof.

12. Apparatus facilitating insertion of a contact lens in a user’s eye, comprising:
a collapsible stand having a substantially conical bellows-like construction of different diameter steps, with an open interior and an opening at a top portion thereof; and
a tubular insertion tool having a cup at a first end thereof shaped to receive said convex surface of a contact lens therein, and a second end thereof shaped and dimensioned to fit in and be securely yet removably held by said opening at a top portion of said stand.

13. Apparatus as recited in claim 12 wherein said stand is open at the bottom thereof so as to be positionable over a portable light source so that light from said portable light source passes through the open interior of said stand, and through said tubular insertion tool to a contact lens supported by said insertion tool cup.

14. Apparatus as recited in claim 12 wherein said tubular insertion tool consists of a simple rubber or plastic tube with an integrally molded cup at said first end thereof.

15. Apparatus as recited in claim 13 in combination with a portable light source which comprises a cellphone having a light source, said open bottom of said stand positioned over said cellphone light source.

16. Apparatus as recited in claim 12 wherein said stand has a bottom connected to a base, and wherein said base includes at least one light source and at least one battery, said at least one light source positioned so that light emanating therefrom passes upwardly through the open interior of said stand, and through said tubular insertion tool to a contact lens supported by said insertion tool cup.

17. Apparatus as recited in claim 16 wherein said at least one light source comprises a plurality of differently colored LEDs.

18. Apparatus as recited in claim 17 in combination a wireless remote control for controlling the operation, including the brightness, of said LEDs.

19. A contact lens tubular insertion tool consisting of a simple silicone rubber tube with an integrally molded cup, shaped at a first end thereof to receive the convex surface of a contact lens therein.

20. A tubular insertion tool as recited in claim 19 in combination with a contact lens having the convex surface thereof received by said insertion tool cup.
